(19) United States
(12) Patent Application Publication
(10) Pub. No.: US 2002/0076415 A1
(43) Pub. Date: Jun. 20, 2002

(54) Hepatitis C virus gene products

(76) Inventors: Jing-Hsiung Ou, San Gabriel, CA (US); Zhenming Xu, Buena Park, CA (US)

Correspondence Address: McCUTCHEN, DOYLE, BROWN & ENERSEN, LLP, Three Embarcadero Center, San Francisco, CA, 94111, US

(21) Appl. No.: 09/736,959

(22) Filed: Dec. 14, 2000

Related U.S. Application Data
(60) Provisional application No. 60/170,835, filed on Dec. 14, 1999.

(51) Int. Cl. A61K 39/29; C12Q 1/70; C07H 21/04; C07K 14/01; C07K 16/08

(52) U.S. Cl. 424/189.1; 435/5; 530/350; 530/388.3; 536/23.72

(57) ABSTRACT

The present invention provides heretofore unidentified proteins and smaller peptides of HCV, which are produced by translation of the HCV core RNA through a frameshifting mechanism. In one embodiment the invention provides a protein that is about 125 to about 161 amino acids long depending on the HCV genotypes. The smaller peptides of the invention range in length from about 13 amino acids to over 50 amino acids. The invention also includes DNA sequences that encode the polypeptides of the invention, antibodies directed against the polypeptides of the invention, and therapeutic compositions including vaccines and anti-viral compositions. Additionally, the invention provides for methods for diagnosing, preventing and treating hepatitis C using the compounds and compositions of the invention.

### RELATED APPLICATION DATA

This application claims the benefit under 35 U.S.C. 119(e) of U.S. Application Ser. No. 60/170,835 filed Dec. 14, 1999, which is herein incorporated by reference.

### FIELD OF THE INVENTION

This invention relates to hepatitis C virus (HCV) and, in particular, to compositions and methods for treating and diagnosing hepatitis C.

### BACKGROUND

Non-A, Non-B hepatitis (NANBH) is a transmissible disease (or family of diseases) that is believed to be virally induced, and is distinguishable from other forms of virus-associated liver disease, such as those caused by hepatitis A virus (HAV), hepatitis B virus (HBV), delta hepatitis virus (HDV), cytomegalovirus (CMV), or Epstein-Barr virus (EBV). Epidemiologic evidence suggests that there may be three types of NANBH: the water-borne epidemic type; the blood or needle associated type; and the sporadically occurring community acquired type. However, the number of causative agents is unknown.

Recently a new viral species, hepatitis C virus (HCV) has been identified as the primary (if not only) cause of blood-associated NANBH (BA-NANBH). Hepatitis C appears to be the major form of transfusion-associated hepatitis in a number of countries, including the United States and Japan. The major problem in this disease is the frequent progression to chronic liver damage (25-55%). There is also evidence implicating HCV in induction of hepatocellular carcinoma.

Thus, a need exists for an effective method for preventing and treating HCV infection: currently, there is none. Further, there is a significant demand for sensitive, specific methods for screening and identifying carriers of NANBH and NANBH-contaminated blood or blood products. Post-transfusion hepatitis (PTH) occurs in approximately 10% of transfused patients, and NANBH accounts for up to 90% of these cases.

Therefore, there is a need for reliable diagnostic and prognostic tools to detect nucleic acids, antigens, and antibodies related to HCV. In addition, there is also a need for effective vaccines and immunotherapeutic agents for the prevention and/or treatment of hepatitis C.

### SUMMARY

The present invention provides heretofore unidentified proteins and smaller peptides of HCV, which are produced by translation of the HCV core RNA through a frameshifting mechanism. In one embodiment the invention provides a protein that is about 125 to about 161 amino acids long depending on the HCV genotypes. The smaller peptides of the invention range in length from about 13 amino acids to over 50 amino acids. The invention also includes DNA sequences that encode the polypeptides of the invention, antibodies directed against the polypeptides of the invention, and therapeutic compositions including vaccines and anti-viral compositions. Additionally, the invention provides for methods for diagnosing, preventing and treating hepatitis C using the compounds and compositions of the invention.

### BRIEF DESCRIPTION OF THE FIGURES

FIG. 1: Analysis of the translational termination and initiation sites of p17. (A) Termination of the p17 sequence in the internal overlapping ORF. pCMV-CC contained the wild-type core protein coding sequence (lane 1), and pCMV-CCmt contained the core protein coding sequence with a premature termination codon in the overlapping ORF (lane 2). The arrow denotes the location of the truncated p17. The HCV sequence in these two DNA constructs was under the control of the T7 promoter and the immediate early promoter of cytomegalovirus. The RNA was synthesized using the T7 RNA polymerase after the plasmids were linearized with XbaI. The translation was carried out at 30° C. for one hour using the following condition: 0.5-1 mg RNA, 10 μl rabbit reticulocyte lysates (Promega), 0.5 μl 1 mM amino acid mixture without methionine, and 50 μCi 35S-methionine (>1000 Ci/mmole, ICN). The proteins synthesized were then analyzed by gel-electrophoresis and autoradiography. The replacement of the reticulocyte lysates with the wheat germ extracts in the translation reaction generated the same results. For the construction of pCMV-CC, the HCV-1 core protein coding sequence was isolated by PCR using the following two primers: sense, CGTGCCCCCGCAAGCTTGCTAG; and antisense, CCGTGGAGTTCTAGACTTGGTTAGGCCGAAGC. The antisense primer contained a termination codon (underlined) at the end of the core protein coding sequence for translation termination. The PCR product was cloned into the XbaI/HindIII site of the pRc/CMV vector (Invitrogen), and the HCV sequence in pCMV-CC was verified by direct sequencing. pCMV-CCmt was identical to pCMV-CC, except that nucleotide 432 of the core protein coding sequence was mutated from T to A and the XbaI-NarI fragment of the vector was removed during the construction. (B) Initiation of the p17 sequence at the core protein initiation codon. Lane 1, translation of the wild-type HCV core protein coding sequence; and lane 2, translation of the core protein sequence fused to the HA-tag. pCMV-HACC contained the HA-tag coding sequence. This plasmid was constructed by inserting the HA coding sequence TACCCATACGACGTCCCAGACTACGCT between the first and the second codons of the core protein coding sequence in pCMV-CC.

FIG. 2: Radiosequencing of p21c and p17. (A) 3H-lysine labeling experiment. The arrow indicates the peak at cycle 11 that was not detected when p17 was sequenced. (B) 3H-threonine-labeling experiment. The translation was carried out using the following condition: 10 mg RNA derived from pCMV-CC, 50 μl wheat germ extracts (Promega), 150 μCi 3H-lysine (or 90 mCi 3H-threonine), 50 μCi 35S-methionine, and 1 mM amino acid mixture minus methionine and lysine (or threonine). p21c and p17 synthesized this way were separated on a 12.5% acrylamide gel. The protein samples were excised from the gel and eluted in 10 mM ammonium bicarbonate containing 75 mg/ml bovine serum albumin (New England Biolabs) at 37° C. overnight. The samples were then subject to Edman degradation and the sequencing cycles were analyzed by scintillation counting.

FIG. 3: Synthesis of the E1 protein from the core-E1 sequence carrying the deletion of nucleotide 30. The DNA plasmid pGEM-CEHA9a, which contained the core-E1 coding sequence with the deletion of nt. 30, was linearized with HindIII and used as the template for the synthesis of the core-E1 RNA. The translation of this RNA using the reticulocyte lysates was carried out as described in the legend to FIG. 1. Lane 1, translation in the absence of microsomal membranes (Promega); lanes 2 and 5, translation in the presence of microsomal membranes; lane 3, same as in lane 2, except that the sample was treated with EndoH; lane 4, same as in lane 3 but without EndoH treatment; lane 6, immunoprecipitation with monoclonal anti-E1 antibody; and lane 7, immunoprecipitation with a control antibody. The EndoH reaction was carried out in the following condition: mix 0.5 μL translational mixture with 1 μl 10× denaturation buffer (New England Biolabs [NEB]), add water to 10 μl, heat at 100° C. for two minutes, then add 1.5 μL 10× W5 buffer (NEB), 1 μl EndoH (NEB) and water to a final volume of 15 μl, and then incubate at 37° C. overnight. The control sample in lane 5 was processed the same way except that EndoH was omitted in the reaction. pGEM-CE1HA9a was constructed by PCR using the following two primers: TTGGAGGCGCTGCCAGGGCCCTGGCGCAT (sense), and GCATTAAAGCTTCCAGCGTAGTCTGGGACGTCGTATGGGTACGCGTCGACGCCGG (antisense). The PCR product was digested with BstX1 and HindIII, and ligated to the 2.8 Kb BstX1-Hindi fragment of pGEM-core9a. This resulted in the creation of pGEM-CE1HA9a. pGEM-core9a contained the deletion of nt. 30 of the core protein coding sequence, which was inserted into the BamH1/HindIII site of the pGEM vector.

FIG. 4: Expression of core and E1 proteins from core and core-E1 sequences carrying nt. 30 deletion. pCDEF-core9a and pCDEF-CE1HA9a contained the core and the core-E1 coding sequences, respectively. The core protein sequence in both plasmids contained the deletion of nt. 30. The expression of the HCV sequences in these two plasmids was under the control of the eEF-1a promoter. These two plasmids were transfected into Huh7 cells, and forty-eight hours after transfection, cells were lysed by a brief sonication in TBS (10 mM Tris-HCl , pH 7.0, 150 mM NaCl) containing 0.1% Nonidet P-40. The cell lysates were then centrifuged at 15,000× g for one minute, and the supernatant was analyzed by Western-blot using either the monoclonal anti-E1 antibody (T. Ohno, M. Mizokami, Methds. Mol. Med. 19, 147 (1998)) or the anti-core antibody (S. -Y. Lo, F. Masiarz, S. B. Hwang, M. M. C. Lai, J. H. Ou, Virology 213, 455 (1995)). Lane 1, cells transfected with pCDEF-core9a; lane 2, cells transfected with pCDEF-CE1HA9a; and lane 3, cells transfected with the control pCDEF vector. For the construction of pCEDF-core9a and pCDEF-CE1HA9a, the BamHI and HindIII (blunt-ended) HCV DNA fragment was isolated from either pGEM-core9a or pGEM-CE1HA9a and subcloned into the BamHI/XbaI (blunt-ended) site of the pCDEF vector (D. W. Kim, T. Harada, I. Saito, T. Miyamura, Gene 134, 307 (1993); W. Lu, S. -Y. Lo, M. Chen, K. -J. Wu, Y. K. T. Fung, J. H. Ou, Virology 263, 134 (1999); S. -Y. Lo, J. H. Ou. Methds. Mol. Med. 19, 325 (1998)).

FIG. 5: Analysis of anti-p17 sera in patients. (A) Immunoprecipitation of p17. The p17 RNA was synthesized from pGEM-core9a with T7 RNA polymerase and translated using the rabbit reticulocyte lysates. The protein was radiolabeled with 35S-methionine. Ten ml of the translational mixture was then incubated with 10 ml of the sera isolated from HCV (+) or HBV (−) patients for radioimmunoprecipitation analysis. (B) Immunoprecipitation of the protein synthesized from the overlapping ORF. A C to G mutation was introduced at nt. 13 of the core protein sequence to create a translation initiation codon for the overlapping ORF. This ORF was then isolated by PCR and cloned into the NdeI/BamH1 site of the pET-3a vector (1098). The synthesis of the RNA and the protein and the radioimmunoprecipitation were conducted as described in (A).

FIG. 6: (A) The putative pseudoknot structure for ribosomal pausing. The structure has a free energy of −48.9 kCal. I and II indicate the two stems. The arrow denotes the base pairings between the sequences of the two loops. (B) Comparison of the HCV sequences in the vicinity of the shift site. The sequences shown represent approximately 95% of the HCV sequences compiled in the HCV database (the HCV database was compiled at the following website: http://s2as02.genes.nig.ac.jp).

FIG. 7: The predicted p17 sequence. The sequence was deduced from the HCV-1 coding sequence (Q. L. Choo, K. H. Richman, J. H. Han, K. Berger, C. Lee, C. Dong, C. Gallegos, D. Coit, R. Medina-Selby, P. J. Barr, A. J. Weiner, D. W. Bradley, G. Kuo, M. Houghton, Proc. Natl. Acad. Sci. USA 88, 2451 (1991)).

FIG. 8: The results of an experiment in which the α-globin coding sequence was used as a reporter and fused to the 5′ end of the HCV core protein coding sequence. The translation of this chimeric sequence produced three major protein products: the globin-core fusion protein (G-core), the globin-17 kDa fusion protein (G-F1) and the globin-small peptide fusion protein (G-F2).

### DEFINITIONS

The term “antibody or antibody molecule” in the various grammatical forms is used herein as a collective noun that refers to a population of immunoglobulin molecules and/or immunologically active portions of immunoglobulin molecules, i.e., molecules that contain an antibody combining site or paratope.

An “antibody combining site” is that structural portion of an antibody molecule comprised of heavy and light chain variable and hypervariable regions that specifically binds antigen.

The phrase “monoclonal antibody” in its various grammatical forms refers to a population of antibody molecules that contain only one species of antibody combining site capable of immunoreacting with a particular epitope. A monoclonal antibody may therefore contain an antibody molecule having a plurality of antibody combining sites, each immunospecific for a different epitope, e.g., a bispecific monoclonal antibody.

Use of the term “having the binding specificity of” indicates that equivalent monoclonal antibodies compete for binding to a preselected target epitope.

The term “nucleotide sequence” refers to a heteropolymer of nucleotides or a sequence of nucleotides. One of skill in the art will readily discern from contextual cues which of the two definitions is appropriate. The terms “nucleic acid” and “polynucleotide” are also used interchangeably herein to refer to a heteropolymer of nucleotides. Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “oligonucleotide fragment” or a “polynucleotide fragment,” “portion,” or “segment” refer to a stretch of nucleotide residues which is long enough to use in polymerase chain reaction (PCR) or various hybridization procedures to identify or amplify identical or related parts of mRNA or DNA molecules.

The term “recombinant,” when used herein to refer to a polypeptide or protein, means that a polypeptide or protein is derived from recombinant (e.g., microbial, mammalian, or insect-based) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

The term “recombinant expression vehicle or vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an N-terminal methionine residue. This residue may or may not be subsequently cleaved from the expressed recombinant protein to provide a final product.

The term “recombinant expression system” means host cells which have stably integrated a recombinant transcriptional unit into chromosomal DNA or carry the recombinant transcriptional unit extrachromosomally. Recombinant expression systems as defined herein will express heterologous polypeptides or proteins upon induction of the regulatory elements linked to the DNA segment or synthetic gene to be expressed. This term also means host cells which have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

The term “polypeptide” refers to a polymer of amino acids and does not refer to a specific length of the molecule. Thus, peptides, oligopeptides, and proteins are included within the definition of polypeptide. This term also does not refer to or exclude post-expression modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring. Moreover, the terms “polypeptide” and “protein” are used interchangeably throughout this specification.

The term “active” refers to those forms of the polypeptide which retain the biologic and/or immunologic activities of any naturally occurring polypeptide.

The term “naturally occurring polypeptide” refers to polypeptides produced by cells that have not been genetically engineered and specifically contemplates various polypeptides arising from post-translational modifications of the polypeptide including, but not limited to, acetylation, carboxylation, glycosylation, phosphorylation, lipidation and acylation.

The term “derivative” refers to polypeptides chemically modified by such techniques as ubiquitination, labeling (e.g., with radionuclides or various enzymes), pegylation (derivatization with polyethylene glycol) and insertion or substitution by chemical synthesis of amino acids such as omithine, which do not normally occur in human proteins.

The term “recombinant variant” refers to any polypeptide differing from naturally occurring polypeptides by amino acid insertions, deletions, and substitutions, created using recombinant DNA techniques. Guidance in determining which amino acid residues may be replaced, added or deleted without abolishing activities of interest, such as cellular trafficking, may be found by comparing the sequence of the particular polypeptide with that of homologous peptides and minimizing the number of amino acid sequence changes made in regions of high homology.

Preferably, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are typically in the range of about 1 to 5 amino acids. The variation allowed may be experimentally determined by systematically making insertions, deletions, or substitutions of amino acids in a polypeptide molecule using recombinant DNA techniques and assaying the resulting recombinant variants for activity.

Alternatively, where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/turnover rate. Further, such alterations can be selected so as to generate polypeptides that are better suited for expression, scale up and the like in the host cells chosen for expression. For example, cysteine residues can be deleted or substituted with another amino acid residue in order to eliminate disulfide bridges.

As used herein, “substantially equivalent” can refer both to nucleotide and amino acid sequences, for example a mutant sequence, that varies from a reference sequence by one or more substitutions, deletions, or additions, the net effect of which does not result in an adverse functional dissimilarity between the reference and subject sequences. Typically, such a substantially equivalent sequence varies from one of those listed herein by no more than about 2% (i.e., the number of individual residue substitutions, additions, and/or deletions in a substantially equivalent sequence, as compared to the corresponding reference sequence, divided by the total number of residues in the substantially equivalent sequence is about 0.02 or less). Such a sequence is said to have 98% sequence identity to the listed sequence. In one embodiment, a substantially equivalent, e.g., mutant, sequence of the invention varies from a listed sequence by no more than 2% (98% sequence identity); in a variation of this embodiment, by no more than 0.5% (99.5% sequence identity); and in a further variation of this embodiment, by no more than 0.1% (99.9% sequence identity). Substantially equivalent, e.g., mutant, amino acid sequences according to the invention generally have at least 98% sequence identity with a listed amino acid sequence, whereas substantially equivalent nucleotide sequence of the invention can have lower percent sequence identities, taking into account, for example, the redundancy or degeneracy of the genetic code. For the purposes of determining equivalence, truncation of the mature sequence (e.g., via a mutation which creates a spurious stop codon) should be disregarded.

Where desired, an expression vector may be designed to contain a “signal or leader sequence” which will direct the polypeptide through the membrane of a cell. Such a sequence may be naturally present on the polypeptides of the present invention or provided from heterologous protein sources by recombinant DNA techniques.

A polypeptide “fragment,” “portion,” or “segment” is a stretch of amino acid residues of at least about 5 amino acids, often at least about 7 amino acids, typically at least about 9 to 13 amino acids, and, in various embodiments, at least about 17 or more amino acids. To be active, any polypeptide must have sufficient length to display biologic and/or immunologic activity.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

The term “activated” cells as used in this application are those which are engaged in extracellular or intracellular membrane trafficking, including the export of neurosecretory or enzymatic molecules as part of a normal or disease process.

The term “purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99.8% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other component normally present in a solution of the same. The terms “isolated” and “purified” do not encompass nucleic acids or polypeptides present in their natural source.

By “pharmaceutically acceptable salt” it is meant those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 1977, 66: 1-19. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters includes formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A. C. S. Symposium Series, and in Edward B. Roche, ed., Bioreversible Carriers in Drug Design, American Pharmaceutical Association and Pergamon Press, 1987, both of which are incorporated herein by reference.

It is well known in the art that modifications and changes can be made in the structure of a polypeptide without substantially altering the biological function of that peptide. For example, certain amino acids can be substituted for other amino acids in a given polypeptide without any appreciable loss of function. In making such changes, substitutions of like amino acid residues can be made on the basis of relative similarity of side-chain substituents, for example, their size, charge, hydrophobicity, hydrophilicity, and the like.

The term “open reading frame,” ORF, means a series of nucleotide triplets coding for amino acids without any termination codons and is a sequence translatable into protein.

The term “expression modulating fragment,” EMF, means a series of nucleotides which modulates the expression of an operably linked ORF or another EMF.

The term “infection” refers to the introduction of nucleic acids into a suitable host cell by use of a virus or viral vector.

The term “transformation” means introducing DNA into a suitable host cell so that the DNA is replicable, either as an extrachromosomal element, or by chromosomal integration.

The term “transfection” refers to the taking up of an expression vector by a suitable host cell, whether or not any coding sequences are in fact expressed.

Each of the above terms is meant to encompasses all that is described for each, unless the context dictates otherwise.

### DETAILED DESCRIPTION OF THE INVENTION

HCV is a Flavi-like virus. The morphology and composition of Flavivirus particles are known, and are discussed by Brinton (1986) THE VIRUSES: THE TOGAVIRIDAE AND FLAVIVIRIDAE (Series eds. Fraenkel-Conrat and Wagner, vol eds. Schlesinger and Schlesinger, Plenum Press), p.327-374. It has been found that portions of the HCV genome are also homologous to pestiviruses. Generally, with respect to morphology, Flaviviruses contain a central nucleocapsid surrounded by a lipid bilayer. Virions are spherical and have a diameter of about 40-50 nm. Their cores are about 25-30 mn in diameter. Along the outer surface of the virion envelope are projections that are about 5-10 nm long with terminal knobs about 2 nm in diameter.

The HCV genome is comprised of RNA. It is known that RNA containing viruses have relatively high rates of spontaneous mutation, i.e., reportedly on the order of 10⁻³ to 10⁻⁴ per incorporated nucleotide. Therefore, there are multiple strains, which may be virulent or avirulent, within the HCV class or species.

It is believed that the genome of HCV isolates is comprised of approximately 9,000 nucleotides to approximately 12,000 nucleotides, and it is possible to differentiate between gene regions, which code for structural proteins such as the envelope (E) and the core (C) proteins. Moreover, the genome comprises genes for non-structural genes components of the virus, such as enzymes, etc. In addition, the genome is believed to be a positive-stranded RNA.

It has been known that the genome can express different proteins but the individual, viral proteins are not that well-known. In accordance with the present invention it has been found that the translation of the HCV core protein RNA produces, in addition to the core protein, an approximately 17 kDa protein called p17 or F1, and a small peptide, F2. The p17 protein encoded by the genomes of various HCV genotypes varies in length from about 125 amino acids to about 161 amino acids.

Due to its small size, the small peptide F2 is not visible unless it is fused to a reporter. The length of this small peptide ranges from 13 amino acids to over 50 amino acids, depending on the genotypes.

Both the 17 kDa novel protein and this small peptide can be produced by ribosomal frameshift during translation or by transcriptional stuttering in the vicinity of codon 10 of the HCV core protein sequence. The 17 kDa protein is derived from the +1/−2 overlapping reading frame and the small peptide is derived from the −1/+2 overlapping reading frame.

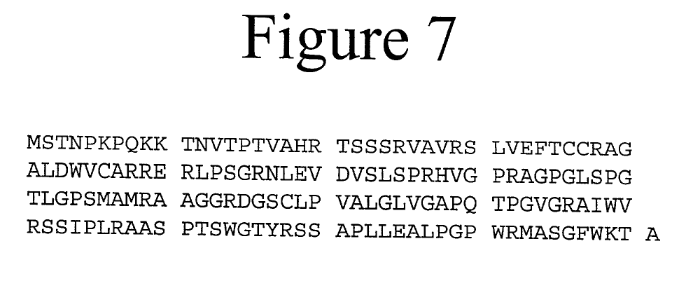

The predicted amino acid sequence of p17 is shown in FIG. 7. This protein is highly basic with a pI value of 12.3. For comparison, the pI value of the HCV core protein is only 11.8 and G-F2. p17 can play a role in viral morphogenesis or, alternatively, regulate the virus-host interactions. p17 and/or G-F2 may serve as new targets for the development of anti-HCV therapies. The prevalence of the anti-p17 antibodies among HCV patients indicates that these peptides may also be used to further improve HCV diagnostic assays.

### Proteins

In accordance with the present invention, it has been discovered that a heretofore unidentified family of hepatitis C virus (HCV) proteins is formed by expression of an overlapping open reading frame in the core protein sequence through a frameshifting mechanism. Such proteins may be formed by transcriptional frameshifting or ribosomal frameshifting, and have been named F proteins (for the frameshifting mechanism by which they are formed). FIG. 7 provides the amino acid sequence, SEQ ID NO:1, for a 161 amino acid, 17 kDa protein. Because this 17 kDa protein is a common protein produced by many genotypes of HCV, the longer proteins of this invention have also been termed p17 proteins. Many of these proteins are also characterized in that they contain a short leader sequence from the core protein. This leader sequence is: SEQ ID NO:22-MSTNPK.

As noted above, the overlapping ORF of the core protein gene sequence of the HCV produces a long protein, p17 (F1), and a short peptide, F2, The longer protein is derived from the +1/−2 overlapping reading frame. As noted above, in some embodiments of the invention, this protein has a length of about 125 amino acids to about 161 amino acids. Exemplary sequences of the p17 protein are sequences of SEQ ID NO:1 to SEQ ID NO:22. On the other hand, the shorter peptide is derived from the −1/+2 overlapping reading frame and, in many genotypes, has a length of about 13 amino acids to about 50 amino acids. F2 molecules from several HCV isolates are comprised of SEQ 40160327_{—}1.DOC NO:22.

The invention further provides a polypeptide including an amino acid sequence that is substantially equivalent to a sequence belonging to the group of sequences consisting of SEQ ID NO: 1 to SEQ ID NO:22.

A variety of methodologies known in the art can be utilized to obtain any one of the isolated polypeptides or proteins of the present invention. At the simplest level, the amino acid sequence can be synthesized using commercially available peptide synthesizers. This is particularly useful in producing small peptides and fragments of larger polypeptides. Fragments are useful, for example, in generating antibodies against the native polypeptide. In an alternative method, the polypeptide or protein is purified from cells which naturally produce the polypeptide or protein. One skilled in the art can readily follow known methods for isolating polypeptides and proteins in order to obtain one of the isolated polypeptides or proteins of the present invention. These include, but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual, Cold Spring Harbor Laboratory, N.Y. (1989); Ausubel, et al., Current Protocols in Molecular Biology, John Wiley & Sons, N.Y. (1989).

The polypeptides and proteins of the present invention can alternatively be purified from cells which have been altered to express the desired polypeptide or protein. One skilled in the art can readily adapt procedures for introducing and expressing either recombinant or synthetic sequences into eukaryotic or prokaryotic cells in order to generate a cell which produces one of the polypeptides or proteins of the present invention. The purified polypeptides can be used in in vitro binding assays which are well known in the art to identify molecules which bind to the polypeptides.

The protein may also be produced by known conventional chemical synthesis. Methods for constructing the proteins of the present invention by synthetic means are known to those skilled in the art. For polypeptides more than about 100 amino acid residues, a number of smaller peptides will be chemically synthesized and ligated either chemically or enzymatically to provide the desired full-length polypeptide. The synthetically-constructed protein sequences, by virtue of sharing primary, secondary or tertiary structural and/or conformational characteristics with proteins may possess biological properties in common therewith. Thus, they may be employed as biologically active or immunological substitutes for natural, purified proteins in screening of therapeutic compounds and in immunological processes for the development of antibodies.

The proteins provided herein also include proteins characterized by amino acid sequences substantially equivalent to those of purified proteins but into which modification are naturally provided or deliberately engineered. For example, modifications in the peptide or DNA sequences can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein.

Other fragments and derivatives of the sequences of proteins which would be expected to retain protein activity in whole or in part and may thus be useful for screening or other immunological methodologies may also be easily made by those skilled in the art given the disclosures herein. Such modifications are intended to be encompassed by the present invention.

### Antibodies

The present invention also provides for antibodies, monoclonal or polyclonal, or fragments thereof, directed against the peptides of the invention such as p17 (F1) and F2. Antibodies against p17 or F2 can be raised by any method known to one of skill in the art. Antibodies may be raised, for example, in an animal such as a rabbit or a mouse against the entire p17 protein or from a peptide fragment or epitope of the protein. Antibodies may also be selected from phage display libraries of antibodies or antibody fragments. Such antibodies, specific for p17 or F2, are useful for the diagnosis of HCV in patients through techniques such as ELISA or other suitable immunochemical methods. Antibodies are useful if, in a standard ELISA assay, they are able to differentiate between HCV-infected and uninfected individuals, or those infected with hepatitis B virus. Also, p17 and F2 antibodies maybe raised that are directed against specific genotypes or genotype classes of HCV. Such antibodies are useful for differentiating between different genotypes.

Antibodies of the invention that specifically bind to p17 or F2 may also be useful as therapeutic agents against HCV. The peptides p17 and F2 can also be used as targets for small molecule therapeutics. Small molecule binding agents can be screened for their ability to bind p17 or F2. Small molecules that interact with p17 or F2 can also be screened for their ability to inhibit the life cycle of HCV.

Thus, in a preferred embodiment, the invention provides antibodies directed against a hepatitis C virus (HCV) core protein, which are elicited by immunizing an animal using one of the partially purified proteins of the invention. As noted above, in some embodiments such antibodies are monoclonal and in other embodiments they are polyclonal.

In general, techniques for preparing polyclonal and monoclonal antibodies as well as hybridomas capable of producing the desired antibody are well known in the art (Campbell, A. M., Monoclonal Antibodies Technology: Laboratory Techniques in Biochemistry and Molecular Biology, Elsevier Science Publishers, Amsterdam, The Netherlands (1984); St. Groth et al., J. Immunol. 35:1-21 (1990); Kohler and Milstein, Nature 256:495-497 (1975)), the trioma technique, the human B-cell hybridoma technique (Kozbor et al, Immunology Today 4:72 (1983); Cole et al., in Monoclonal Antibodies and Cancer Therapy, Alan R. Liss, Inc. (1985), pp. 77-96).

Any animal (mouse, rabbit, etc.) which is known to produce antibodies can be immunized with a peptide, e.g., the p17 protein. Methods for immunization are well known in the art. Such methods include subcutaneous or intraperitoneal injection of the peptide. One skilled in the art will recognize that the amount of the peptide used for immunization will vary based on the animal which is immunized, the antigenicity of the peptide and the site of injection. The peptide that is used as an immunogen may be modified or administered with an adjuvant to increase the peptide's antigenicity. Methods of increasing the antigenicity of a peptide are well known in the art and include, but are not limited to, coupling the antigen with a heterologous protein (such as globulin or β-galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Research, 175:109-124 (1988)).

Hybridomas secreting the desired antibodies are cloned and the class and subclass is determined using procedures known in the art (Campbell, A. M., Monoclonal Antibody Technology: Laboratory Techniques in Biochemistry and Molecular Biology, Elsevier Science Publishers, Amsterdam, The Netherlands (1984)). Techniques described for the production of single chain antibodies (U.S. Pat. No. 4,946,778) can be adapted to produce single chain antibodies to the targeted peptides, e.g., p17 protein or the F2 peptide.

Other methods of producing a monoclonal antibody, a hybridoma cell, or a hybridoma cell culture also are well known. See, for example, the method of isolating monoclonal antibodies from an immunological repertoire as described by Sastry et al. (1989) Proc. Natl. Acad. Sci. USA, 86:5728-5732; and Huse et al. (1989) Science, 246:1275-1281.

For polyclonal antibodies, antibody containing antiserum is isolated from the immunized animal and is screened for the presence of antibodies with the desired specificity using one of the above-described procedures.

The present invention further provides the above-described antibodies in detectably labeled form. Antibodies can be detectably labeled through the use of radioisotopes, affinity labels (such as biotin, avidin, etc.), enzymatic labels (such as horseradish peroxidase, alkaline phosphatase, etc.) fluorescent labels (such as FITC or rhodamine, etc.), paramagnetic atoms, etc. Procedures for accomplishing such labeling are well-known in the art, for example, see (Stemberger, L. A. et al., J. Histochem. Cytochem. 18:315 (1970); Bayer, E. A. et al, Meth. Enzym. 62:308 (1979); Engval, E. et al., Immunol. 109:129 (1972); Goding, J. W. J. Immunol. Meth. 13:215 (1976)).

If desired, the antibodies can also be used to make anti-idiotype atibodies which in turn can be humanized as is known in the art to prevent immunological responses. Humanized monoclonal antibodies offer particular advantages over murine monoclonal antibodies, particularly insofar as they can be used therapeutically in humans. Specifically, human antibodies are not cleared from the circulation as rapidly as “foreign” antigens, and do not activate the immune system in the same manner as foreign antigens and foreign antibodies.

The antibody of the invention can also be a fully human antibody such as those generated, for example, by selection from an antibody phage display library displaying human single chain or double chain antibodies such as those described in de Haard, H. J. et al. (1999) J. Biol. Chem. 274:18218-30 and in Winter, G. et al. (1994) Annu. Rev. Immunol. 12:433-55.

The labeled antibodies of the present invention can be used for in vitro, in vivo, and in situ assays to identify cells or tissues in which a fragment of the receptor molecule of interest is expressed. The antibodies also may be used directly in therapies or other diagnostics. The present invention further provides the above-described antibodies immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir, D. M. et al., Handbook of Experimental Immunology 4th Ed., Blackwell Scientific Publications, Oxford, England, Chapter 10 (1986); Jacoby, W. D. et al., Meth. Enzym. 34 Academic Press, N.Y. (1974)). The immobilized antibodies of the present invention can be used for in vitro, in vivo, and in situ assays as well as for immuno-affinity purification of receptors for which the antibodies have a binding affinity. The antibodies can also be used in research to further elucidate the functioning of the signaling pathways in activation of cells by growth factors, hormones, cytokines or the like.

Antisera titer may be established through several means known in the art, such as by dot blot and density analysis, and also by precipitation of radiolabeled peptide-antibody complexes using a protein, secondary antisera, cold ethanol or charcoal-dextran followed by activity measurement with a gamma counter. If desired, the highest titer antisera may be purified on affinity columns. For example, a peptide such as the p17 protein may be coupled to a commercially available resin and used to form an affinity column. Antiserum samples may then be passed through the column so that antibodies to the peptide bind (via the peptide) to the column. These bound antibodies are subsequently eluted, collected and evaluated for determination of titer and specificity.

An additional way to determine whether a monoclonal antibody has the specificity of a monoclonal antibody of the invention is to determine the amino acid residue sequence of the CDR regions of the antibodies in question. Antibody molecules having identical, or functionally equivalent, amino acid residue sequences in their CDR regions have the same binding specificity. Methods for sequencing polypeptides are well known in the art. This does not suggest that antibodies with distinct CDR regions cannot bind to the same epitope.

Exemplary antibodies for use in the present invention include intact immunoglobulin molecules, substantially intact immunoglobulin molecules and those portions of an immunoglobulin molecule that contain the paratope, including those portions known in the art as Fab, Fab′, F(ab′)₂ and F(v), and also referred to as antibody fragments. The Fab fragment, lacking Fc receptor, is soluble, and affords therapeutic advantages in serum half life, and diagnostic advantages in modes of using the soluble Fab fragment. The preparation of a soluble Fab fragment is generally known in the immunological arts and can be accomplished by a variety of methods.

For example, Fab and F(ab′)₂ portions (fragments) of antibodies are prepared by the proteolytic reaction of papain and pepsin, respectively, on substantially intact antibodies by methods that are well known. See for example, U.S. Pat. No. 4,342,566 to Theofilopolous and Dixon. Fab′ antibody portions also are well known and are produced from F(ab′).sub.2 portions followed by reduction of the disulfide bonds linking the two heavy chain portions as with mercaptoethanol, and followed by alkylation of the resulting protein mercaptan with a reagent such as iodoacetamide.

### Polynucleotides and Nucleic Acids of the Invention

The present invention also provides nucleotide sequences that encode the proteins of the invention, for example, the p17 protein and the F2 protein. An example of such sequences is the core protein RNA sequence of HCV. Such sequences may be used as vaccines for prevention and treatment of HCV. Thus, the present invention also includes the administration of a nucleic acid vector capable of expressing the polypeptides of the inventions into an animal, wherein the nucleic acid molecule can elicit an immune response in, and preferably immunize, an animal against the expressed protein expressed from the nucleic molecule, and therefore HCV. In one embodiment of this procedure, naked DNA is introduced into an appropriate cell, such as a muscle cell, where it produces protein that is then displayed on the surface of the cell, thereby eliciting a response from host cytotoxic T-lymphocytes (CTLs). This can provide an advantage over traditional immunogens wherein the elicited response comprises specific antibodies. Specific antibodies are generally strain-specific and cannot recognize the corresponding antigen on a different strain. CTLs, on the other hand, are specific for conserved antigens and can respond to different strains expressing a corresponding antigen (Ulmer et al., “Heterologous protection against influenza by injection of DNA encoding a viral protein,” Science 259:1745-1749, 1993; Lin et al., “Expression of recombinant genes in myocardium in vivo after direct injection of DNA,” Circulation 82:2217-21, 1990); Wolff et al., “Long-term persistence of plasma DNA and foreign gene expression in mouse muscle,” Human Mol. Gen. 1:363-69, 1992)

Upon introduction of the naked vector construct into the animal's cell, the construct is then able to express the nucleic acid molecule (typically a gene) that it carries, which gene preferably comprises one (or more) of the HCV proteins of the invention. Accordingly, upon expression of the desired peptide, an immune response is elicited from the host animal. Preferably, the immune response includes CD8.sup.+CTLs able to respond to different strains that exhibit a form of the desired peptide.

Thus, in a preferred embodiment the invention provides a DNA vaccine for immunizing a mammal against hepatitis C comprising a DNA sequence that encodes for at least one of the HCV proteins of the invention in a pharmacologically acceptable carrier.

The invention also provides the complement of the polynucleotides of the invention including a nucleotide sequence that has at least about 95%, more typically at least about 99%, and even more typically at least about 99.5%, sequence identity to a polynucleotide encoding a polypeptide recited above. The polynucleotide can be DNA (genomic, cDNA, amplified, or synthetic) or RNA. Methods and algorithms for obtaining such polynucleotides are well known to those of skill in the art and can include, for example, methods for determining hybridization conditions which can routinely isolate polynucleotides of the desired sequence identities.

The polynucleotides of the invention can be DNA molecules that are isolated from nucleic acid sequences present in the plasma of an HCV infected patient. The process of isolation includes the steps of isolating viral particles from the patient's plasma, extracting and purifying the viral nucleic acid sequences, and then cloning the desired DNA molecule via a Polymerase Chain Reaction (PCR) technique.

A polynucleotide according to the invention can be joined to any of a variety of other nucleotide sequences by well-established recombinant DNA techniques (see Sambrook J et al. (1989) Molecular Cloning: A Laboratory Manual, Cold Spring Harbor Laboratory, N.Y.). Useful nucleotide sequences for joining to polynucleotides include an assortment of vectors, e.g., plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication function in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The present invention further provides recombinant constructs comprising a nucleotide sequence of the invention, or a fragment thereof. The recombinant constructs of the present invention comprise a vector, such as a plasmid or viral vector, into which a nucleic acid of the invention or a fragment thereof is inserted, in a forward or reverse orientation. In the case of a vector comprising one of the ORFs of the present invention, the vector may further comprise regulatory sequences, including for example, a promoter, operably linked to the ORF.

Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTI, pSG (Stratagene) pSVK3, pBPV, pMSG, pSVL (Pharmacia).

### Therapeutic Compositions

The compounds of the invention, i.e., proteins, polynuclotides, peptides, antibodies, etc. may be used with or without other compositions and procedures for the treatment of diseases. Thus, in a preferred embodiment the invention provides a composition comprising one of the earlier-described proteins and an excipient, diluent or carrier. Alternatively, the composition is an anti-viral composition that comprises a compound that binds to the proteins of the invention. In another embodiment, such a composition is a vaccine for immunizing a mammal against hepatitis C comprising at least one of the above proteins in a pharmacologically acceptable carrier. Additionally, the compounds of the invention may be combined with pharmaceutically acceptable excipients, and optionally sustained-release matrices, such as biodegradable polymers, to form therapeutic compositions.

A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

When used in the above or other treatments, a therapeutically effective amount of one of the compounds of the present invention may be employed in pure form or, where such forms exist, in pharmaceutically acceptable salt form. By a “therapeutically effective amount” of the compound of the invention is meant a sufficient amount of the compound to treat HCV. It will be understood, however, that the total daily usage of the compounds of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved.

The compositions of the present invention can be used in the form of salts derived from inorganic or organic acids. These salts include but are not limited to the following: acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxy-ethansulfonate (isothionate), lactate, maleate, methanesulfonate, nicotinate, 2-naphthalenesulfonate, oxalate, pamoate, pectinate, persulfate, 3-phenylpropionate, pcrate, pivalate, propionate, succinate, tartrate, thiocyanate, phosphate, glutamate, bicarbonate, p-toluenesulfonate and undecanoate. Water or oil-soluble or dispersible products are thereby obtained.

Examples of acids which may be employed to form pharmaceutically acceptable addition salts include such inorganic acids as hydrochloric acid, sulphuric acid and phosphoric acid and such organic acids as maleic acid, succinic acid and citric acid. Other salts include salts with alkali metals or alkaline earth metals, such as sodium, potassium, calcium or magnesium or with organic basis. Preferred salts of the compositions of the invention include phosphate, tris and acetate.

The total daily dose of the compositions of this invention administered to a human or lower animal may range from about 0.001 to about 1 mg/kg of patients body mass/day. If desired, the effective daily dose may be divided into multiple doses for purposes of administration; consequently, single dose compositions may contain such amounts or submultiples thereof to make up the daily dose.

Alternatively, a compound of the present invention may be administered as a pharmaceutical composition containing the compound of interest in combination with one or more pharmaceutically acceptable excipients. A pharmaceutically acceptable carrier or excipient refers to a non-toxic solid, semi-solid or liquid filler, diluent, encapsulating material or formulation auxiliary of any type. The compositions may be administered parenterally, intracistemally, intravaginally, intraperitoneally, topically (as by powders, ointments, drops or transdermal patch), rectally, or bucally. The term “parenteral” as used herein refers to modes of administration which include intravenous, intramuscular, intraperitoneal, intrasternal, subcutaneous and intraarticular injection and infusion.

Pharmaceutical compositions for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Topical administration includes administration to the skin or mucosa, including surfaces of the lung and eye. Compositions for topical administration, including those for inhalation, may be prepared as a dry powder which may be pressurized or non-pressurized. In non-pressurized powder compositions, the active ingredient in finely divided form may be used in admixture with a larger-sized pharmaceutically-acceptable inert carrier comprising particles having a size, for example, of up to 100 micrometers in diameter. Suitable inert carriers include sugars such as lactose. Desirably, at least 95% by weight of the particles of the active ingredient have an effective particle size in the range of 0.01 to 10 micrometers.

Alternatively, the composition of the invention may be pressurized and contain a compressed gas, such as nitrogen or a liquified gas propellant. The liquified propellant medium and indeed the total composition is preferably such that the active ingredient does not dissolve therein to any substantial extent. The pressurized composition may also contain a surface active agent, such as a liquid or solid non-ionic surface active agent or may be a solid anionic surface active agent. It is preferred to use the solid anionic surface active agent in the form of a sodium salt.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The present compositions in liposome form can contain, in addition to a conjugate of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

Total daily dose of the compositions of the invention to be administered to a human or other mammal host in single or divided doses may be in amounts, for example, from 0.0001 to 300 mg/kg body weight daily and more usually 1 to 300 mg/kg body weight.

It will be understood that agents which can be combined with the compounds of the present invention for the diagnosis, inhibition or treatment of hepatitis C are not limited to those listed above, but include, in principle, any agents useful for the diagnosis or treatment of hepatitis C.

### Methods for Treating Hepatitis C

The invention also provides for methods for treating various disease states. Generally, the methods comprise administering to a subject the compounds and compositions of the invention described above. In preferred embodiments, as a person of ordinary skill in the art would recognize, the compounds and compositions are administered in a therapeutically effective amount and with a pharmaceutically acceptable carrier as discussed above.

Thus, in one embodiment the invention provides a method of preventing hepatitis C, the method comprising administering a vaccine of the invention to a mammal in an amount effective to stimulate the production of a protective antibody. The vaccine may be a DNA vaccine of the invention or a vaccine that comprises one of the proteins of the invention. In another embodiment, the invention provides a method of treating hepatitis C in a human subject, the method comprising administering an anti-viral composition of the invention to the human subject.

The dosage ranges for the administration of the compounds and compositions of the invention depend upon the form of the compound or composition, and its potency, and are amounts large enough to produce the desired effect in which hepatitis C symptoms are ameliorated. The dosage should not be so large as to cause adverse side effects, such as hyperviscosity syndromes, pulmonary edema, congestive heart failure, and the like. Generally, the dosage will vary with the age, condition, sex and extent of HCV infection in the patient and can be determined by one of skill in the art. The dosage also can be adjusted by the individual physician in the event of any complication.

The compounds and compositions of the invention including monoclonal antibodies, polypeptides, vaccines, anti-viral agents, and derivatives thereof can be administered intravenously, intraperitoneally, intramuscularly, subcutaneously, intracavity, transdermally, topically, intraocularly, orally, intranasally and can be delivered by peristaltic means.

The compositions of this invention can be administered intravenously, as by injection of a unit dose, for example. The term “unit dose” when used in reference to a composition of the present invention refers to physically discrete units suitable as unitary dosage for the subject, each unit containing a predetermined quantity of active material calculated to produce the desired therapeutic effect in association with the required diluent; i.e., carrier, or vehicle.

### Detection Methods

The compounds of the invention also are suitable for detection of HCV. In a preferred embodiment the invention provides a method for analyzing HCV antigen in a sample that comprises contacting the antibodies of the invention with the sample under conditions suitable for the antibodies to form a complex with a hepatitis C virus antigen protein, and detecting the complex and thereby determining whether HCV antigen is in the sample. In another embodiment, the invention provides a method for detecting hepatitis C virus (HCV) antibodies in a sample, comprising contacting one or more of the proteins of the invention with the sample under conditions which allow binding of the protein with antibodies directed against an HCV antigen in the sample to form an antigen-antibody complex, and then detecting the antigen-antibody complex.

In various embodiments, the above methods are carried out by solid phase-immunoassay and/or are characterized by using an enzyme or isotope substance as a label. Thus, in one embodiment, the invention provides an enzyme-linked immunosorbent assay (ELISA) for detecting hepatitis C virus antibodies in samples, which comprises the following steps: a) coating one or more of the proteins of the invention onto a solid phase, b) contacting a sample suspected of containing HCV antibodies with the protein coated onto the solid phase under conditions which allow the formation of an antigen-antibody complex, c) adding a anti-human antibody conjugated with an enzyme label to be captured by the antigen-antibody complex bound to the solid phase, and d) detecting the captured label and determining whether the sample has HCV antibodies. In one embodiment of this method, the ELISA is such that the solid phase is a microtiter plate. In another embodiment, the solid phase comprises horseradish peroxidase.

Immunological techniques such as immunostaining and ELISA are described in, for example, Receptor Binding Techniques, Methods in Molecular Biology. 106. ed. M. Keen. Humana Press, 1999; Brooks et al. (1998) Cell 92:391-400; Brooks et al. (1996) Cell 85:683-693; and Brooks et al. (1993) J. Cell. Biol. 122:1351-1359.

The labels used for detecting the antibody-antigen complexes may comprise a moiety, which is detectable label such as a fluorochrome, a radioactive tag, paramagnetic heavy metal or diagnostic dye.

### EXAMPLES

The following examples serve to illustrate the present invention. Selection of analytical methods as well as the concentration of reagents, temperatures, and the values of other variables are only to exemplify application of the present invention and are not to be considered limitations thereof.

In particular the example establish that the core protein gene sequence of HCV expresses heretofore unidentified proteins. These proteins are expressed by an overlapping open reading frame in the core protein gene sequence through various frameshifting mechanisms. The examples also strongly indicate that these proteins are produced during natural HCV infection in patients establishing that the detection of antisera to these proteins can be used to diagnose infection of a patient with HCV.

Previous studies suggest that, in addition to the 21 kDa core protein, p21, other proteins may be expressed from the core protein gene of the HCV isolate both in vitro and in mammalian cells (S. -Y. Lo, M. Selby, M. Tong, J. H. Ou, Virology 199, 124 (1994); S. -Y. Lo, F. Masiarz, S. B. Hwang, M. M. C. Lai, J. H. Ou, Virology 213, 455 (1995); R. B. Ray, L. M. Lagging, K. Meyer, R. Ray, J. Virol. 70, 4438 (1996). The expression of these proteins is thought to be independent of the downstream E1 envelope protein sequence (S. -Y. Lo, M. Selby, M. Tong, J. H. Ou, Virology 199, 124 (1994); S. -Y. Lo, F. Masiarz, S. B. Hwang, M. M. C. Lai, J. H. Ou, Virology 213, 455 (1995); S. -Y. Lo. M. J. Selby, J. H. Ou, J Virol. 70, 5177 (1996)). The identity of these proteins, however, has been unclear.

### Example I

### Overlapping Open Reading Frame

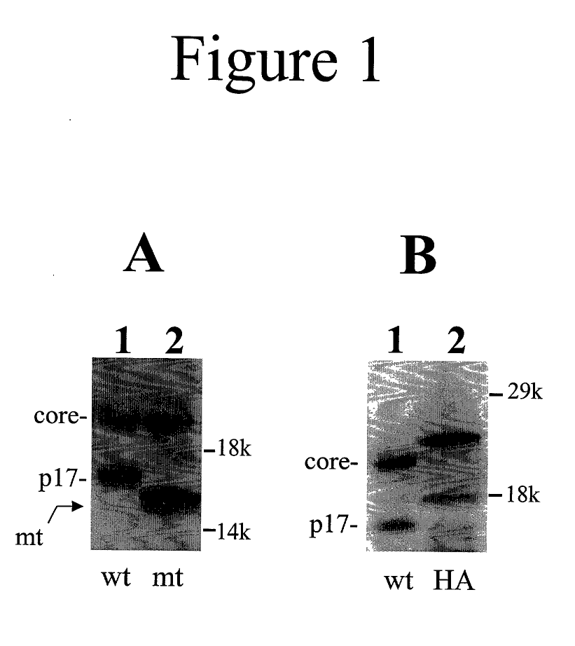

It has been discovered by inspection of the HCV-1 core protein coding sequence that a +1 overlapping coding sequence spans from nucleotide (nt.) 5 to nt. 485. This overlapping coding sequence, which lacks an ATG codon near its 5′ end, has a coding capacity of 160 amino acids. To investigate whether the previously unknown protein is derived from this overlapping open reading frame (ORF), nt. 432 of the HCV-1 coding sequence was converted from T to A to create a premature termination codon in ORF. This mutation removed the last eighteen codons of the ORF without affecting the core protein coding sequence. If the protein of this invention was derived from the ORF, then this mutation should reduce the size of the protein by approximately 2 kDa without affecting the synthesis of the core protein. This was indeed the case. As shown in FIG. 1A, the translation of the wild-type HCV-1 core protein RNA in vitro using the rabbit reticulocyte lysates generated the 21 kDa core protein (p21c) and a 17 kDa protein, which can be called p17. In contrast, the translation of the HCV-1 RNA containing the mutation generated p21 c and a smaller 15 kDa protein. This result strongly indicates that p17 was derived from the overlapping ORF.

### Example II

### Another Peptide From the Core Protein Sequence

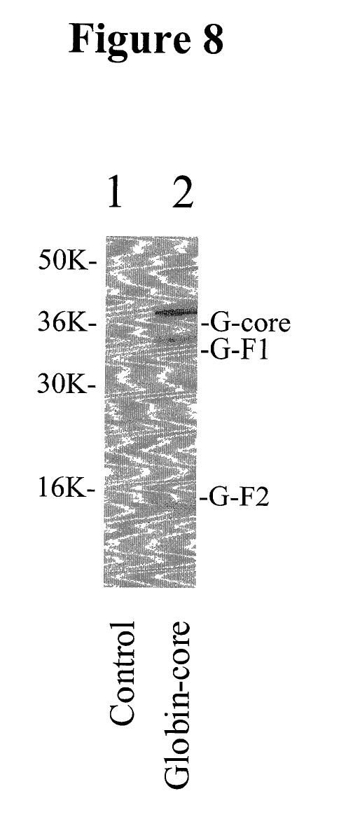

As shown above, the translation of the HCV core protein RNA produces the core protein and the 17 kDa protein. It was also found that this translation also produces a small peptide. Due to its small size, this small peptide is not visible unless it is fused to a reporter. The α-globin coding sequence was used as a reporter and fused to the 5′ end of the HCV core protein coding sequence. The translation of this chimeric sequence produced three major protein products: the globin-core fusion protein (G-core), the globin-17 kDa fusion protein (G-F1) and the globin-small peptide fusion protein (G-F2). See FIG. 8. The length of this small peptide ranges from 13 amino acids to over 50 amino acids, depending on the genotypes. Both the 17 kDa novel protein and this small peptide can be produced by frameshift during translation or by transcriptional stuttering in the vicinity of codon 10 of the HCV core protein sequence. The 17 kDa protein is derived from the +1/−2 overlapping reading frame and the small peptide is derived from the −1/+2 overlapping reading frame.

### Example III

### Frameshifting

Although the overlapping ORF contains three methionine residues, none of them resides near the 5′-end of its coding sequence. Thus, if p17 was derived from the ORF, it must be synthesized by initiation from a non-ATG codon or by frameshift after translation initiation from the ATG codon of the core protein sequence. To distinguish between these two possibilities, the coding sequence of the HA-tag was fused in frame to the 5′-end of the core protein coding sequence. If p17 was synthesized from a non-ATG codon located near the 5′-end of the core protein coding sequence, then its synthesis would unlikely be affected by the sequence of the HA-tag. On the contrary, if p17 was synthesized from the ATG codon of the core protein coding sequence, then the HA-tag should increase the p17 size correspondingly. As shown in FIG. 1B, the HA-tag increased the size of both p21and p17 correspondingly. The presence of the HA-tag in both p21c and p17 was verified by immunoprecipitation using an anti-HA antibody (data not shown). Thus, the results shown in FIG. 1 indicated that p17 was synthesized from the initiation codon of the core protein sequence and terminated at the termination codon of the overlapping ORF in the +1 reading frame.

### Example IV

### +1 Ribosomal Frameshift

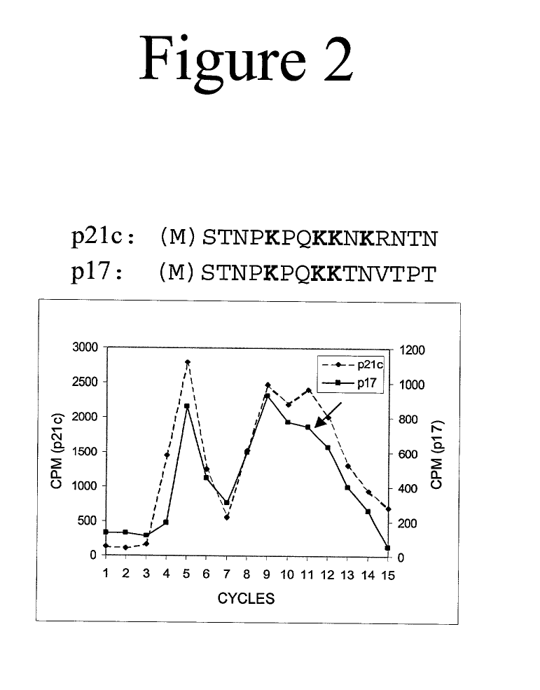

The results shown in FIG. 1 suggest a scenario of ribosomal frameshift during the synthesis of p17. Since our previous results indicated that a mutation in codon 9 of the HCV-1 core protein coding sequence would significantly reduce the efficiency of p17 expression (S. -Y. Lo, M. Selby, M. Tong, J. H. Ou, Virology 199, 124 (1994)), the frameshift site for the synthesis of p17 likely occurred in the vicinity of this codon. To investigate this possibility, we decided to perform the radiosequencing experiment. The HCV-1 core protein RNA was translated in vitro using wheat germ extracts in the presence of 35S-methionine and 3H-lysine. Both p21c and p17 that were synthesized were purified from the protein gel and subjected to radiosequencing. Few 35S -methionine counts were detected in the first sequencing cycle whether p21 c or p17 was sequenced (data not shown), indicating the loss of the initiator methionine residue, possibly caused by the amino-terminal peptidase activity in the wheat germ extracts. As shown in FIG. 2A, the sequencing of p21c generated three distinct 3H-lysine peaks at sequencing cycles 5, 9 and 11. There are four lysine residues at amino acid 6, 9, 10 and 12 of the p21c sequence. Since lysines 9 and 10 were not expected to be resolved into separate peaks in the sequencing reaction, the p21 c sequencing result was in agreement with the p21c sequence minus the initiator methinonine residue. The sequencing of p17 generated 3H-lysine peaks resembling those generated from the sequencing of p21c, except that the peak at cycle 11 had largely disappeared. Since the 3H-lysine peaks at cycles 9 and 10 did not appear to be affected, this result indicated that the sequences of p21c and p17 diverged after amino acid 10. Due to the presence of ten contiguous A nucleotides between codons 8-11 (see below), a +1 ribosomal frameshift between these codons would fuse the first 10 amino acids of the core protein sequence to the ORF sequence (FIG. 2A). This would generate a p17 sequence lacking the lysine residue at position 12.

To further investigate whether p17 was synthesized by a +1 ribosomal frameshift, a different amino acid, 3H-threonine, was used to label p21c and p17 for radiosequencing. As shown in FIG. 2B, the sequencing of 3H-threonine-labeled p21c generated two peaks at cycles 2 and 14. This result is again in agreement with the predicted p21c sequence minus the initiator methionine residue. The sequencing of p17, in contrast, generated four 3H-threonine peaks at cycles 2, 10 and 13. These peaks were consistent with the p17 sequence generated by a +1 ribosomal frameshift between codons 2 and 10 of the core protein coding sequence. Taken together, the results shown in FIG. 2 indicate that p17 contains a hybrid sequence derived from the core protein and the +1 ORF. Its synthesis starts from the core protein initiation codon and, after reading eight to ten codons, frameshifts into the +1 ORF.

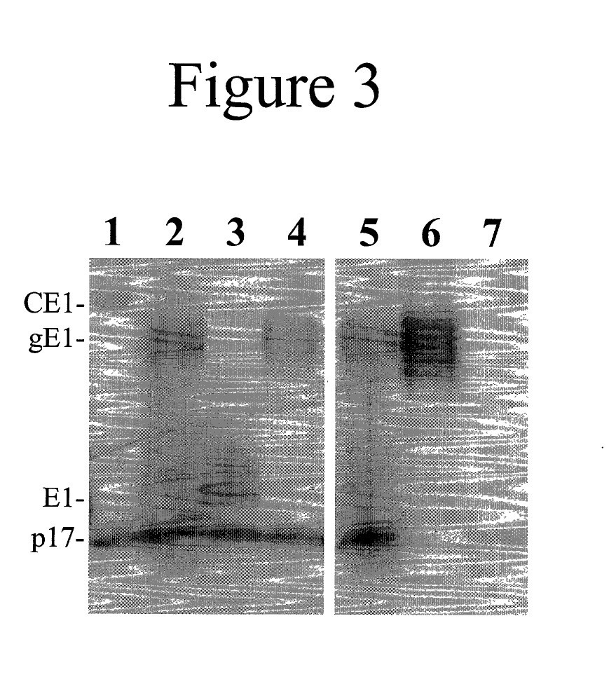

To ensure that the HCV-1 core protein gene sequence could indeed direct ribosomal frameshift in cells and also to investigate the mechanism of this ribosomal frameshift, a single A residue was deleted from the stretch of 10 A's located at nt. 24-33 of the HCV-1 core protein coding sequence for the expression studies. Due to this deletion, the first ten codons of the core protein sequence were fused to the +1 ORF. Thus, p17 should be the predominant protein produced from the mutated core or core-E1 sequence and any synthesis of the full-length core protein or E1 would imply a −1 or a +2 ribosomal frameshift. The experiment was first conducted in vitro using the rabbit reticulocyte lysates. The translation of the mutated core protein sequence produced predominantly p17 with little core protein when the proteins were radiolabeled with 35S-methionine (data not shown; also see below). However, when the mutated core-E1 sequence was translated, p17 as well as a minor protein species with a molecular weight of approximately 40 kDa was detected (FIG. 3, lane 1). We believe this 40 kDa protein was the C-E1 fusion protein for the following reasons. First, the combined molecular weights of core and E1 proteins are approximately 40 kDa. Second, if the translation was carried out in the presence of microsomal membranes, this 40 kDa protein was converted to a protein band with a molecular weight of approximately 33 kDa (FIG. 3, lane 2), which is the molecular weight of the glycosylated E1. Third, the incubation with EndoH converted the 33 kDa protein to a 20 kDa protein (FIG. 3, lanes 3 and 4), which is similar to the molecular weight of the nonglycosylated E1 protein. And finally, the 33 kDa glycoprotein could be immunoprecipitated by a monoclonal anti-E1 antibody but not by a control antibody (FIG. 3, lanes 5-7).

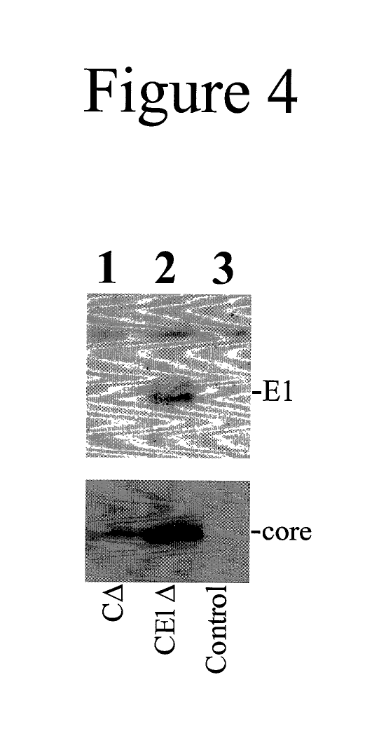

The synthesis of C-E1 in this experiment was most likely achieved by ribosomal framneshift during the synthesis of p17. Note that the core protein signal could not be detected in the presence of microsomal membranes (FIG. 3, lanes 2-5), which separated core and E1 proteins in an equal molar ratio. The lack of the core protein signal was due to the presence of only one methionine residue in the core protein sequence after the removal of its initiator methionine residue. In comparison, the E1 protein has eight methionine residues, which greatly increased the sensitivity of its detection since the proteins were labeled with 35S-methionine. The core protein could be detected by Western-blot analysis (see below). Next Huh7 cells, a well-differentiated human hepatoma cell line, were used as the host cells for the expression of the mutated core sequence and the core-E1 sequence. The proteins expressed were then analyzed by Western-blot. As shown in FIG. 4, the core protein can be expressed from both sequences and the E1 protein can be expressed from the mutated core-E1 sequence. This result is consistent with the in vitro results shown in FIG. 3 and indicated that the HCV sequence could correct the single-nucleotide deletion to direct the synthesis of the core protein and E1. This result is indicative of a −1 (or +2) ribosomal frameshift.

### Example V

### Protein Synthesis During Natural HCV Infection

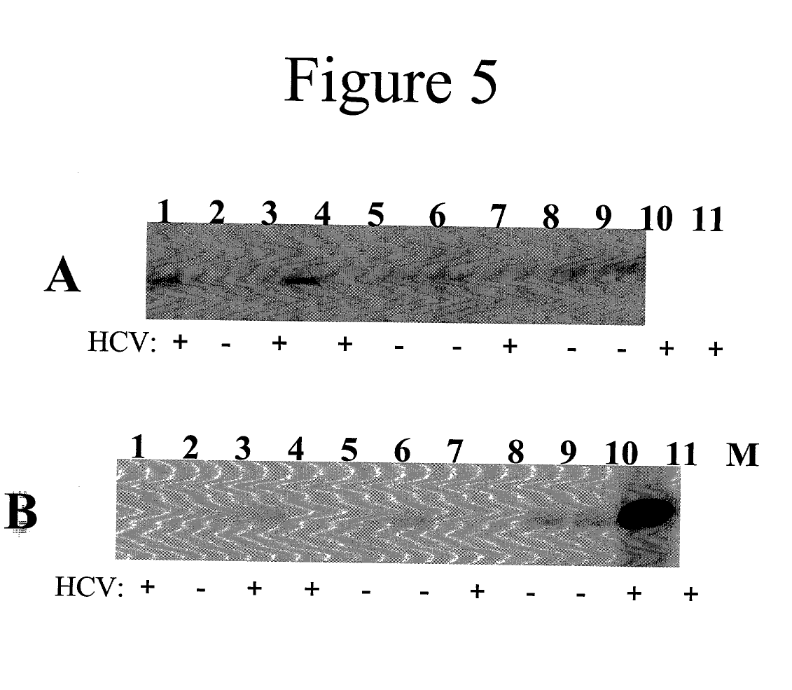

As shown above, p17 could be produced by the HCV-1 sequence in vitro and in mammalian cells. It was decided to determine whether it could be synthesized during natural HCV infection in patients and, therefore it was decided to test the presence of p17 reactive antibodies in HCV patients. The core protein sequence with the deletion of nt. 30 was used for this study. As mentioned above, this nucleotide deletion fused the first ten codons of the core protein sequence to the +1 ORF to create p17. p17 was synthesized from this mutated sequence, radiolabeled with 35S-methionine, and immunoprecipitated with the sera isolated from six HCV patients and five HBV patients. As shown in FIG. 5A, while none of the HBV sera immunoprecipitated p17, five of the six HCV sera clearly reacted with p17. Because p17 contains a short leader sequence from the core protein, it is possible that the immunoreactivity seen in FIG. 5A was due to the cross-reaction between the anti-core antibodies and p17. To rule out this possibility, a C to G mutation was created at nt. 13 of the core protein coding sequence. This mutation creates an ATG codon in the overlapping ORF. This “complete” ORF was then isolated by the polymerase chain reaction (PCR) and inserted into the pET-3a expressing vector under the T7 promoter control. The RNA was then synthesized from this expressing vector, translated using the rabbit reticulocyte lysates, radiolabeled with 35S-methionine, and immunoprecipitated with either the HCV sera or the HBV sera. As shown in FIG. 5B, similar to FIG. 5A, while none of the HBV sera reacted with the protein derived from this internal overlapping ORF, all of the HCV sera reacted with this protein, albeit weakly. Thus, the results shown in FIG. 3 establish that p17 is produced during natural HCV infection in patients. Such detection of antisera to p17 can be used to diagnose infection of a patient with HCV.

### The p17 Protein Family

A survey of the HCV sequences compiled in an online database indicates that p17, which has a length of 161 amino acids, is conserved in 100% of the HCV genotype 1a sequences (Table I). Its truncated forms ranging from 125 amino acids to 154 amino acids in length are also conserved in the great majority of the HCV sequences reported. As shown in Table I, p17 sequence is truncated predominantly at amino acid 143 and 125 for genotypes 1b and 2a, respectively. Thus, the length of p17 appears to be genotype-specific. The constraint imposed by two overlapping coding sequences is likely the reason why the HCV core protein coding sequence has the lowest synonymous substitution rate among all the HCV genes (Y. Ina, M. Mizokami, K. Ohba, T. Gojobori, J. Mol. Evol. 38, 50 (1994)). Table II provides exemplary sequences for a number of the analyzed sequences. From the disclosure provided, one of skill in the art can identify the p17 gene product from any particular HCV genomic sequence. Thus, the sequences provided in Table II are provided to illustrate the method of determining a p17 sequence from a particular genomic HCV sequence.

### The Frameshifting Mechanism

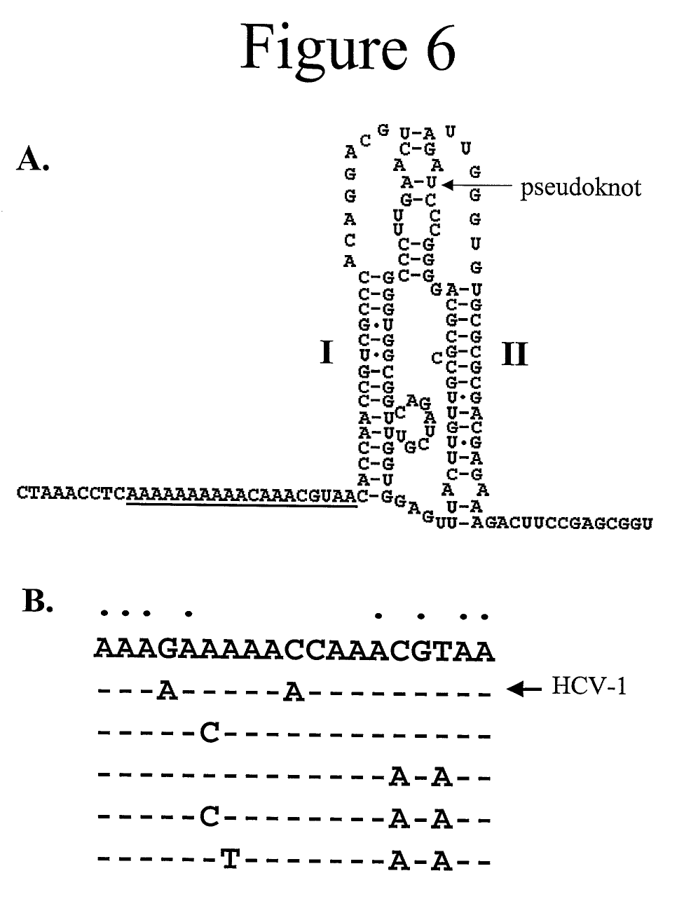

The eukaryotic ribosomal frameshift signal typically includes a shift site and a pseudoknot structure (P. J. Farabaugh, Annu. Rev. Genet. 30, 507 (1996)). The latter is thought to serve as the ribosomal pausing site to facilitate ribosomal frameshift (P. J. Farabaugh, Annu. Rev. Genet. 30, 507 (1996)). For HCV, too, a stable secondary structure, which contains two stems and a pseudoknot structure formed between two hairpin loops, can be predicted from the 3′-flanking sequence of the shift site (FIG. 6A). Conceivably, this secondary structure or its variant(s) mediate(s) ribosomal pausing to facilitate ribosomal frameshift. The sequence located in the vicinity of the shift site is A-rich. For HCV-1, a stretch of 10 As could be identified (FIG. 6). These 10 As spread from codon 8 to codon 11 and hence the ribosomal frameshift could theoretically take place after reading codon 8, 9 or 10 and still generate the same p17 sequence due to the repetition of the sequence. The sequence in the vicinity of the shift site is highly conserved among the reported HCV sequences (FIG. 6B). This conservation may be due to its double coding function and/or its role in directing ribosomal frameshift. The consensus sequence of the shift site for -1 ribosomal frameshift is X XXY YYZ, where X can be any of the four nucleotides, Y is A or U, and Z is A, U or C (I. Brierley, A. J. Jenner, S. C. Inglis, J. Mol. Biol. 227, 463 (1992)). The stretch of 10 As located in the vicinity of the shift site contains this consensus sequence, which might be the reason why the synthesis of core and E1 proteins could be rescued from the sequences that contained a single-nucleotide deletion (FIGS. 3 and 4).

An internal ribosomal entry site (IRES) which encompasses most of the 5′ noncoding region and the first nine codons of the core protein sequence has been identified in the HCV genome. This IRES mediates the translation of the HCV polyprotein sequence but has no effect on the synthesis of p17 (M. J. Selby, Q. L. Choo, K Berger, G. Kuo, E. Glazer, M. Eckart, C. Lee, D. Chien, C. Kuo, M. Houghton, J. Gen. Virol. 74, 1103 (1993), data not shown). Thus, the ribosomal frameshift signal which directs the synthesis of p17 acts independently of the HCV IRES. How the HCV ribosomal frameshift signal directs the synthesis of p17 remains unknown. Nevertheless, this signal appears to be rather unusual as it could direct both +1 and −1 (or +2) ribosomal frameshift.

TABLE I
The length of p17 encoded by the genomes of different HCV

| genotypesa,b | | | | | | | |
|---|---|---|---|---|---|---|---|
| Genotypes | 125 a.a.^{c} | 130 a.a.^{c} | 139 a.a.c^{c} | 143 a.a.^{c} | 154 a.a.^{c} | 161 a.a.^{c} | others^{d} |
| 1a (16) | | | | | | 100% (16) | |
| 1b (102) | 1% (1) | | 3% (3) | 80% (82) | | 7% (7) | 9% (9) |
| 2a (24) | 80% (19) | | 4% (1) | 8% (2) | | | 8% (2) |
| 2b (7) | | | | | 71% (5) | 29% (2) | |
| 3a (11) | | | | 36% (4) | 46% (5) | 18% (2) | |
| 3b (12) | 42% (5) | | 50% (6) | | | | 8% (1) |
| 4 (24) | 58% (14) | 13% (3) | | | 8% (2) | 17% (4) | 4% (1) |
| 5 (2) | 50% (1) | | | | | 50% (1) | |
| 6 (10) | 30% (3) | 30% (3) | | | | 40% (4) | |
| ^{a}235 HCV sequences compiled in (The HCV database was compiled at the following website: http://s2as02.genes.nig.ac.jp) were analyzed. The genotyping was based on the procedures of Ohno and Mizokami (T. Ohno, M. Mizokami, Methds. Mol. Med. 19, 147 (1998)). Several of the HCV sequences could be grouped into more than one genotype. For these sequences, their grouping was arbitrary. Twenty-seven HCV sequences could not be genotyped by the procedures | | | | | | | |
| # of Ohno and Mizokami and were not considered. | | | | | | | |

^{b}Numbers in parenthesis indicate the total number of HCV sequences.
^{c}The initiation codon of the core protein was considered amino acid 1. Therefore, amino acid 11 was the first amino acid after ribosomal frameshift.
^{d}The termination codon was found at other nonconserved locations.

TABLE II
Sequences of exemplary p17 sequences. Representative p17
sequences are shown for each class of genotype. GenBank
accession numbers are given in parenthesis for the genomic
sequences from which the p17 protein sequence was identified.

Genotype 1a
SEQ ID NO: 2 (AF011753: 161aa)
MSTNPKPQRKPNVTPTVAHRTSSSWVAVRSLVEFTCCRAGALDWVCARRGRLPSGRNLEVDVSL
SPRHVGPPAOPGLSPGTLGPSMAMRVAOGWDOSCLPVALGLAGAPQTPGVGPAIWVRSSTPLEA
ASPTSWGTYRSSAPLLEALPGPWRMASGFWKTA
SEQ ID NO:3 (M74804: 161aa)
MSTNPKPQRKPNVTPTVAHRTSSSRVAVRSLVEFTCCRAGALDWVCARRGRLPSGRNLEVDVSP
SPRLVDPRAGPGLSPGTLGPSMAMRAAGGRDGSCLPVALGLAGAPQTPGVORAIWVRSSIPLEA
ASPTSWOTYRSSAPLLEALPEPWRMASGFWKTA
Genotype 1b
SEQ ID NO:4 (AF054250: 143aa)
MSTNPKPQRKPNVTPTAAHRTSSSEAVVRSLVEFTCCPAGAPGWVCARLGRLPSORNLVEODNL
SQRLAXPRAGPGLSPGTLGPSMAMRAWGGQDGSCHPAAPGLVGAPRTPGVGRVTWVRSSIPLEA
ASPISWGTFRSSAPP
SEQ ID NO:5 (D10934: 143aa)
MSTNPKPQRKPNVTLTAAERTSSSEAVARSLVEFTCCEAGAPGWVCARLGRLPSGRNLVEGDNL
SPRLAGPRAGPGLSPGTLGPSMAMPAWGGQDGSCHPAALOLVGAPMTPGVGRVIWVRSSIPSHA
AS PTSWGTFRS SAPP
SEQ ID NO:6 (D49758: 139aa)
MSTNPKPQRQPEETPTVAHRTSSSRAAVRSWVEFTCCRAGALDWVCARLGRLPNGPSPEAGVSP
FQRLAARPAVPGVSLGTHGPCMEMRAAGGQGGSCLPAAPAHRGAQTTPGVGPATWVRSSIPSLA
ASPTSWGTSPS
SEQ ID NO:7 (L02836: 161aa)
MSTNPKPQRKPNVTPTAAHRTSSSEAVVRSLVEFTCCRAGAPGWVCARLGRLPSGRNLVEGDNL
SPRLADPPAGPGLSPGILGPSMANRALGGQDGSCHPAAPGLVGAPRTPGVGRVIWVRSSIPSHA
ASPTSWGTFRSSAPPWGALPGPWHMVSGFWRTA
SEQ ID NO:8 (M74809: 143aa)
MSTNPKPQRKPNVTPTAAHRTSSSPAVVRSLVEFTCCRAGAPGWVCARLGRLPSGRNLVEGDNL
SPRLASPPAGPGLSPGTPGPSMANRAWGGQDGSCHPAAPGLVGAPKTPGVGRVIWVRSSIPSHA
ASPTSWGTFRSSAPP
SEQ ID NO:9 (S78528: 143aa)
MSTNPKPQRKPNVTPTAAHRTSSSPAVVRSSVEFTCCRAGALGWVCARLGRLPSGRNLVEGDNL
SPRLADPRAGPGLSPGTLGPSMAMRAWGGQDGSCHPAAPGLVGAPRTPGVGRVIWVRSSIPSHA
ASPTSWGIFRSSAPP
SEQ ID NO:10 (U10199: 143aa)
MSTNPKPQRKPNVTPTAAHRTSSSPAVVRSLVEFTCCPAGAPGWVCARPGRLQSGRNLVEGDNL
SPRLANPRAGPGLSPGILGPSMATRAWGGQDGSCHPAALGLIGAPRTPGVGRAIWVRSSIPSRA
ASPTSWGTSRSSVPP
SEQ ID NO: 11(U16362: 143aa)
MSTNPKPQRKPNVTPTAAHRILSSRAVVRSLVEFTCCRAGAPGWVCARLGRLPSGRNLVEGDSL
SPRLAGPPAGPGLSPGTLGPSMAMPAWGGQDGSCHPAAPGLVGAPRTPGVSRVIWVRSSTPSHA
ASPTSWGTFRSSAPP
SEQ ID NO:12 (X61595: 143aa)
MSTNPKPQRKPNVTPTAAHRTSSSPAVARSLVEFTCFPAGAPGWVCARLGRLPSGRNLVEGDNL
SPRLASPPAGPGLSPGTLGPSMAMRVWGGQDGSCHPVAPGLVGAPRTPOVGRVIWVRSSIPSHA
ASPTPWGTPRSSAPP
Genotype 2a
SEQ ID NO:13 (D00944: 125aa)
MSTNPKPQRKPKETPTVAHKTLSFRAAARSLAEYTCCRAGAPGWVCARQGRLRSGPSHVEGASP
SLRIGAPLANPGENQDTPGPYTGMRDSAGQDGSCPPEVPVPLGAPMTPGIGPATWVRSSIP
SEQ ID NO:14 (L29587: 125aa)
MSTNPKPQRKPNVTPTAALWTLSSQAVVRSLAEFTCCPAGAPGWVCARLGRLRSGRNLVGGANL
SPRRAEPRADPGRSPGILGPFTAMRAVGGQGGSCPLAXLGRLGAPMIPGGDPATWVRSSIP
Genotype 2b
SEQ ID NO:15 (D49755: 154aa)
MSTNPKPQRKPKETPTVAHRTLSSRAAARSLAEYTCCPAGALGWVCARRGRLPNGPSRVEGASP
SPKIGATPASPGDVQDIPGPCMGMRASDGQGGSCPPEGLALHGAPLTPGISRVIWVRSSIPSLA
ALPTSWGIFPSSAPLLVALIPELSRMA
Genotype 3a
SEQ ID NO:16 (D14309: 154aa)
MSTLPKPQRKPKETPSVHRTSSSRVADRSLVEYTCCRAGAHDWVCARRVKLLNGHSLADDDGSL
SPRRVGAKAGPGLSPGTLGPSMVTRAAGGQDGSCPRAAPVHLQAQMTPGDGPAIWIKSSIPLRA
DSPTSWGTSRSSALPWEASQEPSRMA
Genotype 3b
SEQ ID NO:17 (D49750: 139aa)
MSTLPKPQRKPKETPTAGHRTLSSQAAVRSLVEFTYYHAGAPSWVCVQYARLPSGRNLAVGVNP
SPCHAEPPAGPGPSPGTLGPYTGMRAAGGQDGSCPRAALAPRGAQTTPGVDPAIWVRSSIPSHA
DSPTSWGTFRS
Genotype 4
SEQ ID NO:18 (D37848: 125aa)
MSTLPKPQRKPKETPTVAQWTSSSRAAARSWVEFTCYRAGARDWVCARRGRLPNGPSPEAGASP
YQRPAGRPAVAGLSPATPGPYTEMRAAGGQDGFCPPVVLVRVGAQMTPGEGPAIWVRSSTP
SEQ ID NO:19 (D88473: 125aa)
MSTLPKPQRKPKETPTVAQWTSSSRVAVRSLAEFTCCPAGAPGWVCARRERLPSDPSPEAGANL
YQRPASPPAGTGLSPDILGIJFMETRAAGGQVGSCPPAAPGHIGAPMTPGIDPGTWVRSSIP
Genotype 5
SEQ ID NO:20 (AF064490: 125aa)
MSTNPKPQRKPKETPTAAHRTSSSRAVVRSLVEFTCCRAGALGWVCAQLGRLQNGRNPVDGVSL
SPRPSPPAGPGVNPGTLGPFMPMRASGGQGGCSPPEALIGLIGAPMTPGGNRATWVRSSIP
Genotype 6
SEQ ID NO:21 (D88476: 125aa)
MSTLPKPQRKPKETPTVAQWTLSSRVAVRSLAEFTCCRAGAPGWVCARQERLPSDPSLEAGANL
YQRPASLPAGTGLSPDTLGLFMETPAAGGQVGSCPPVAPGHIGAPMTPGVDPGIWVRSSIP

What is claimed is:

1. An isolated and purified protein of the hepatits C virus that is formed by expression of an overlapping open reading frame in the core protein gene sequence through a frameshifting mechanism.

2. The protein of claim 1 wherein the frameshifting mechanism is ribosomal frameshifting.

3. The protein of claim 1 wherein the frameshifting mechanism is transcriptional frameshifting.

4. The protein of claim 1 wherein the amino acid sequence of the protein comprises SEQ ID NO: 1.

5. The protein of claim 1 wherein the protein is derived from the +1/−2 overlapping reading frame.

6. The protein of claim 5 wherein the protein has a length of about 125 amino acids to about 161 amino acids.

7. The protein of claim 1 wherein the protein is derived from the −1/+2 overlapping reading frame.

8. The protein of claim 7 wherein the protein has a length of about 13 amino acids to over 50 amino acids.

9. The protein of claim 1 wherein the protein has an amino acid sequence selected from the group consisting of SEQ ID NO:2 to SEQ ID NO:22.

10. A vaccine for immunizing a mammal against hepatitis C comprising at least one protein of claim 1 in a pharmacologically acceptable carrier.

11. A method of preventing hepatitis C, the method comprising administering the vaccine of claim 10 to a mammal in an amount effective to stimulate the production of a protective antibody.

12. A DNA vaccine for immunizing a mammal against hepatitis C comprising a DNA sequence that encodes for at least one protein of claim 1 in a pharmacologically acceptable carrier.

13. A method of preventing hepatitis C, the method comprising administering the vaccine of claim 12 to a mammal in an amount effective to stimulate the production of a protective antibody.

14. A composition comprising the protein of claim 1 and an excipient, diluent or carrier.

15. A method of preventing hepatitis C, the method comprising administering the composition of claim 14 to a mammal in an amount effective to stimulate the production of a protective antibody.

16. An isolated polypeptide prepared by genetic engineering wherein said polypeptide consists of an amino acid sequence selected from the group consisting of SEQ ID NO: 1 to SEQ ID NO:22.

17. An anti-viral composition comprising a compound that binds to the protein of claim 1.

18. A method of treating hepatitis C in a human subject, the method comprising administering the anti-viral composition of claim 17 to the human subject.

19. Antibodies directed against a hepatitis C virus (HCV) core protein which are elicited by immunizing an animal using a partially purified protein of claim 1.

20. The antibodies of claim 19 wherein the antibodies are monoclonal.

21. The antibodies of claim 19 wherein the antibodies are polyclonal.

22. A method for analyzing HCV antigen in a sample comprising contacting said antibodies of claim 19 with said sample under conditions suitable for said antibodies to form a complex with a hepatitis C virus (HCV) antigen protein, and detecting said complex and thereby determining whether HCV antigen is in said sample.

23. A method for detecting hepatitis C virus (HCV) antibodies in a sample, comprising contacting the protein of claim 1 with said sample under conditions which allow binding of said protein with antibodies directed against an HCV antigen in said sample to form a antigen-antibody complex, and then detecting said antigen-antibody complex.

24. The method of claim 23 wherein the method is carried out by solid phase-immunoassay.

25. The method of claim 23 characterized by using an enzyme or isotope substance as a label.

26. An enzyme-linked immunosorbent assay (ELISA) for detecting hepatitis C virus (HCV) antibodies in samples, which comprises:
a) coating the protein of claim 1 onto a solid phase,
b) contacting a sample suspected of containing HCV antibodies with said polypeptide coated onto the solid phase under conditions which allow the formation of an antigen-antibody complex,
c) adding an anti-human antibody conjugated with an enzyme label to be captured by said antigen-antibody complex bound to the solid phase, and
d) detecting the captured label and determining whether the sample has HCV antibodies.

27. The ELISA of claim 26 wherein the solid phase is a microtiter plate.

28. The ELISA of claim 26 wherein the solid phase comprises horseradish peroxidase.


SEQUENCE LISTING (22 sequences)

SEQ ID NO: 1 - protein, 161 aa, Hepatitis C Virus
  Exemplary P17 sequences
MSTNPKPQKKTNVTPTVAHRTSSSRVAVRSLVEFTCCRAGALDWVCARRERLPSGRNLEVDVSLSPRLVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

SEQ ID NO: 2 - protein, 161 aa, Hepatitis C Virus
  Exemplary P17 sequences
MSTNPKPQRKPNVTPTVAHRTSSSWVAVRSLVEFTCCRAGALDWVCARRGRLPSGRNLEVDVSLSPRHVGPRAGPGLSPGTLGPSMAMRVAGGWDGSCLPVALGLAGAPQTPGVGRAIWVRSSIPLRAASPTSWGTYRSSAPLLEALPGPWRMASGFWKTA

SEQ ID NO: 3 - protein, 161 aa, Hepatitis C Virus
  Exemplary P17 sequences
MSTNPKPQRKPNVTPTVAHRTSSSRVAVRSLVEFTCCRAGALDWVCARRGRLPSGRNLEVDVSPSPRLVDPRAGPGLSPGTLGPSMAMRAAGGRDGSCLPVALGLAGAPQTPGVGRAIWVRSSIPLRAASPTSWGTYRSSAPLLEALPEPWRMASGFWKTA

SEQ ID NO: 4 - protein, 143 aa, Hepatitis C Virus
  Exemplary P17 sequences
MSTNPKPQRKPNVTPTAAHRTSSSRAVVRSLVEFTCCRAGAPGWVCARLGRLPSGRNLVEGDNLSQRLAXPRAGPGLSPGTLGPSMAMRAWGGQDGSCHPAAPGLVGAPRTPGVGRVTWVRSSIPLHAASPISWGTFRSSAPP

SEQ ID NO: 5 - protein, 143 aa, Hepatitis C Virus
  Exemplary P17 sequences
MSTNPKPQRKPNVTLTAAHRTSSSRAVARSLVEFTCCRAGAPGWVCARLGRLPSGRNLVEGDNLSPRLAGPRAGPGLSPGTLGPSMAMRAWGGQDGSCHPAALGLVGAPMTPGVGRVIWVRSSIPSHAASPTSWGTFRSSAPP

SEQ ID NO: 6 - protein, 139 aa, Hepatitis C Virus
  Exemplary P17 sequences
MSTNPKPQRQPEETPTVAHRTSSSRAAVRSWVEFTCCRAGALDWVCARLGRLPNGPSPEAGVSPFQRLAARRAVPGVSLGTHGPCMEMRAAGGQGGSCLPAAPAHRGAQTTPGVGPATWVRSSIPSLAASPTSWGTSPS

SEQ ID NO: 7 - protein, 161 aa, Hepatitis C Virus
  Exemplary P17 sequences
MSTNPKPQRKPNVTPTAAHRTSSSRAVVRSLVEFTCCRAGAPGWVCARLGRLPSGRNLVEGDNLSPRLADPRAGPGLSPGILGPSMAMRALGGQDGSCHPAAPGLVGAPRTPGVGRVIWVRSSIPSHAASPTSWGTFRSSAPPWGALPGPWHMVSGFWRTA

SEQ ID NO: 8 - protein, 143 aa, Hepatitis C Virus
  Exemplary P17 sequences
MSTNPKPQRKPNVTPTAAHRTSSSRAVVRSLVEFTCCRAGAPGWVCARLGRLPSGRNLVEGDNLSPRLASPRAGPGLSPGTPGPSMAMRAWGGQDGSCHPAAPGLVGAPKTPGVGRVIWVRSSIPSHAASPTSWGTFRSSAPP

SEQ ID NO: 9 - protein, 143 aa, Hepatitis C Virus
  Exemplary P17 sequences
MSTNPKPQRKPNVTPTAAHRTSSSRAVVRSSVEFTCCRAGALGWVCARLGRLPSGRNLVEGDNLSPRLADPRAGPGLSPGTLGPSMAMRAWGGQDGSCHPAAPGLVGAPRTPGVGRVIWVRSSIPSHAASPTSWGIFRSSAPP

SEQ ID NO: 10 - protein, 143 aa, Hepatitis C Virus
  Exemplary P17 sequences
MSTNPKPQRKPNVTPTAAHRTSSSRAVVRSLVEFTCCRAGAPGWVCARPGRLQSGRNLVEGDNLSPRLANPRAGPGLSPGILGPSMATRAWGGQDGSCHPAALGLIGAPRTPGVGRAIWVRSSIPSRAASPTSWGTSRSSVPP

SEQ ID NO: 11 - protein, 143 aa, Hepatitis C Virus
  Exemplary P17 sequences
MSTNPKPQRKPNVTPTAAHRILSSRAVVRSLVEFTCCRAGAPGWVCARLGRLPSGRNLVEGDSLSPRLAGPRAGPGLSPGTLGPSMAMRAWGGQDGSCHPAAPGLVGAPRTPGVSRVIWVRSSTPSHAASPTSWGTFRSSAPP

SEQ ID NO: 12 - protein, 143 aa, Hepatitis C Virus
  Exemplary P17 sequences
MSTNPKPQRKPNVTPTAAHRTSSSRAVARSLVEFTCFRAGAPGWVCARLGRLPSGRNLVEGDNLSPRLASPRAGPGLSPGTLGPSMAMRVWGGQDGSCHPVAPGLVGAPRTPGVGRVIWVRSSIPSHAASPTPWGTFRSSAPP

SEQ ID NO: 13 - protein, 125 aa, Hepatitis C Virus
  Exemplary P17 sequences
MSTNPKPQRKPKETPTVAHKTLSFRAAARSLAEYTCCRAGAPGWVCARQGRLRSGPSHVEGASPSLRIGAPLANPGENQDTPGPYTGMRDSAGQDGSCPPEVPVPLGAPMTPGIGPATWVRSSIP

SEQ ID NO: 14 - protein, 125 aa, Hepatitis C Virus
  Exemplary P17 sequences
MSTNPKPQRKPNVTPTAALWTLSSQAVVRSLAEFTCCRAGAPGWVCARLGRLRSGRNLVGGANLSPRRAEPRADPGRSPGILGPFTAMRAVGGQGGSCPLAXLGRLGAPMIPGGDPATWVRSSIP

SEQ ID NO: 15 - protein, 154 aa, Hepatitis C Virus
  Exemplary P17 sequences
MSTNPKPQRKPKETPTVAHRTLSSRAAARSLAEYTCCRAGALGWVCARRGRLPNGPSRVEGASPSPKIGATPASPGDVQDIPGPCMGMRASDGQGGSCPPEGLALHGAPLTPGISRVIWVRSSIPSLAALPTSWGIFPSSAPLLVALPELSRMA

SEQ ID NO: 16 - protein, 154 aa, Hepatitis C Virus
  Exemplary P17 sequences
MSTLPKPQRKPKETPSVAHRTSSSRVADRSLVEYTCCRAGAHDWVCARRVKLLNGHSLADDGSLSPRRVGAKAGPGLSPGTLGPSMVTRAAGGQDGSCPRAAPVHLGAQMTPGDGPAIWVKSSIPLRADSPTSWGTSRSSALPWEASQEPSRMA

SEQ ID NO: 17 - protein, 139 aa, Hepatitis C Virus
  Exemplary P17 sequences
MSTLPKPQRKPKETPTAGHRTLSSQAAVRSLVEFTYYHAGAPSWVCVQYARLPSGRNLAVGVNPSPGHAEPRAGPGPSPGTLGPYTGMRAAGGQDGSCPRAALAPRGAQTTPGVDPAIWVRSSIPSHADSPTSWGTFRS

SEQ ID NO: 18 - protein, 125 aa, Hepatitis C Virus
  Exemplary P17 sequences
MSTLPKPQRKPKETPTVAQWTSSSRAAARSWVEFTCYRAGARDWVCARRGRLPNGPSPEAGASPYQRRAGRRAVAGLSPATPGPYTEMRAAGGQDGFCPPVVLVRVGAQMTPGEGPAIWVRSSTP

SEQ ID NO: 19 - protein, 125 aa, Hepatitis C Virus
  Exemplary P17 sequences
MSTLPKPQRKPKETPTVAQWTSSSRVAVRSLAEFTCCRAGAPGWVCARRERLPSDPSPEAGANLYQRRASPRAGTGLSPDILGLFMETRAAGGQVGSCPPAAPGHIGAPMTPGIDPGIWVRSSIP

SEQ ID NO: 20 - protein, 125 aa, Hepatitis C Virus
  Exemplary P17 sequences
MSTNPKPQRKPKETPTAAHRTSSSRAVVRSLVEFTCCRAGALGWVCAQLGRLQNGRNPVDGVSLSPRRASPRAGPGVNPGTLGPFMPMRASGGQGGCSPPEALGLIGAPMTPGGNRATWVRSSIP

SEQ ID NO: 21 - protein, 125 aa, Hepatitis C Virus
  Exemplary P17 sequences
MSTLPKPQRKPKETPTVAQWTLSSRVAVRSLAEFTCCRAGAPGWVCARQERLPSDPSLEAGANLYQRRASLRAGTGLSPDTLGLFMETRAAGGQVGSCPPVAPGHIGAPMTPGVDPGIWVRSSIP

SEQ ID NO: 22 - protein, 6 aa, Hepatitis C Virus
  Leader sequence
MSTNPK